(19) United States
(12) Patent Application Publication
(10) Pub. No.: US 2013/0065314 A1
(43) Pub. Date: Mar. 14, 2013

(54) ALGAL TRANSFORMATION SYSTEMS, COMPOSITIONS AND METHODS

(71) Applicant: Trevor Wayne MacMillan, Lethbridge (CA)

(72) Inventor: Trevor Wayne MacMillan, Lethbridge (CA)

(73) Assignee: PHYTOCENTRIC BIOTECH INC., Vancouver (CA)

(21) Appl. No.: 13/594,259

(22) Filed: Aug. 24, 2012

Related U.S. Application Data
(60) Provisional application No. 61/527,352, filed on Aug. 25, 2011.

(51) Int. Cl. C12N 15/74; C12N 1/12; C12N 1/13

(52) U.S. Cl. 435/471; 435/257.2; 435/257.1; 435/257.6

(57) ABSTRACT

One aspect of the invention relates to a method for introducing a cargo molecule into an algal cell, the method comprising preparing a composition comprising the cargo molecule and a cell penetrating peptide (CPP) and exposing the algal cell to the composition. The method may also comprise treating the algal cell to disrupt a cell wall of the algal cell. One aspect of the invention relates to a method for transforming an algal cell, the method comprising preparing a composition comprising a nucleic acid molecule and a cell penetrating peptide and exposing the algal cell to the composition. In some embodiments, the CPP is Transportan, Penetratin, an HIV Tat fragment, or a polyarginine, or a variant thereof.

### RELATED APPLICATION

This application claims priority from U.S. Provisional Patent Application No. 61/527352 filed on Aug. 25, 2011 which is hereby incorporated by reference.

### TECHNICAL FIELD

The present invention relates to molecular biology, and more specifically to methods, compositions and systems for introducing molecules into algal cells.

### BACKGROUND

The world is currently facing increasing energy demands and rising energy prices. At the same time, serious environmental concerns are being raised. Algae are considered to be one of the solutions and have attracted a large amount of private and government based investment in research to develop algal renewable chemicals and biofuels. It has been shown that algae is useful in biofuel production [1], bioremediation of wastewater [2], agricultural food production [3], CO₂ sequestering and therapeutic drug manufacturing [4, 5]. Algae are ideally suited for these purposes because they cost very little to cultivate, have a high growth rate and can be grown in a sustainable manner using marginal land and wastewater sources [6]. All algae require to grow is sunlight, CO₂ and a few micronutrients. Microalgae oil yield can be as high as 58,700 L/ha, or 30% oil by weight of algal biomass. In contrast, oil yields of traditional agricultural oil crops such as canola are about 1,190 L/ha. This indicates that biodiesel produced from algae oil can result in 10-20 times more end product than traditional agricultural oil crops [7]. In addition, a new line of pharmaceutical research uses algae for the production of vaccines and for the expression of biological proteins used in the treatment of diseases such as diabetes and multiple sclerosis [9, 10].

Efforts to genetically manipulate algal cells, as required for many research and commercial applications, are limited by problems encountered in the delivery of heterologous molecules (e.g., nucleic acid molecules, probes, proteins, or some other biomolecules) into algal cells. Methods and compositions developed for transforming bacteria cells, animal cells, or plant cells are typically not readily capable of transforming algal cells. Also, many genetic tools developed in bacterial, animal or plant species cannot be used directly in algae. There are many biological differences between algal cells and bacterial cells, animal cells, or plant cells. Many physiological, morphological, biochemical and molecular characteristics of algae are different from plants and animals. In particular, the cell wall and cell membrane of algal cells present two barriers to the entry of heterologous molecules into algal cells. Also, the growth conditions and culture media for algae are different from the growth conditions and culture media for bacterial, animal or plant cells.

Even between algae and plants, there are significant differences. In contrast to plant cell walls which are primarily made up of cellulose, xylan, and lignin, algal cell walls may contain either polysaccharides or a variety of glycoproteins. For example, many green algae species (e.g., Chlamydomonas reinhardtii) have cell walls primarily made up of hydroxyproline-rich glycoproteins. Also, lignin is primarily found in plant cell walls whereas it is rarely found in algal cells. The variation in chemical composition and/or thickness of the cell wall in algae and plants could affect transformation rates. Additionally, life cycles, reproduction, and growth conditions of many algae species are different from those of plant species. The many differences between algae and plant species are consistent with genomic and evolutionary studies which have shown that algae diverged from plant species over a billion years ago.

A number of different methods have been developed to genetically manipulate algae due to their resistance to standard DNA transformation techniques [11-13]. These methods for transforming algal cells are generally based on particle bombardment or electroporation. These methods are complicated and require expensive specialized equipment. Additionally, these methods are not easily scalable, tend to show high variability, and may produce cellular damage and/or contamination. For example, particle bombardments of algal cells tend to cause damage to the algal cells; however, for the transformation to be successful, the affected algal cell must survive the life-threatening damage and resume cell division.

There is a general desire for alternative systems, methods and compositions to transform algal cells.

### BRIEF DESCRIPTION OF DRAWINGS

In drawings which show non-limiting embodiments of the invention:

FIG. 1 is a schematic representation of a method according to an example embodiment of the present invention.

FIG. 2 is a schematic representation of a method according to an example embodiment of the present invention.

FIG. 3 is a graphic representation of a protocol according to an example embodiment of the present invention.

FIG. 3A is a schematic diagram illustrating the ble marker used on one example embodiment of the present invention.

FIG. 4 shows growth curves of C.reinhardtii based on OD values at 680 nm.

FIG. 5 shows graphs showing determination of minimal inhibitory concentration of Zeocin™ for C. reinhardtii.

FIG. 6 is a schematic presentation of differences in growth curves due to sensitivity of non-transformed C. reinhardtii (Nit1-305,cw-15) mutant in SGII-(KNO₃) medium in comparison to the growth in SG-II(NH₄NO₃) medium. The plots are based on (i) OD values at 680 nm and (ii) cell count.

FIGS. 7-9 are examples of pictures of successful transformation showing transformed colonies on selection plates. Each picture contains text which describes the algae type, plasmid name with gene insert and the transformation treatment.

FIG. 10 shows examples of gel electrophoresis results of PCR screening of Chlamydomonas reinhardtii (nit1-305,cw15) transformed colonies.

FIG. 11 shows examples of gel electrophoresis results of the PCR screening of transformant colonies resulted from transformation of C. reinhardtii (Nit1-305,cw-15).

FIGS. 12-14 are example images of 1% agarose gel showing gel mobility shift assay results of various cell penetrating peptides (CPPs) with DNA.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

In one aspect of the invention, there is provided a method for introducing a cargo molecule into an algal cell. In some embodiments, the cargo molecule is introduced into an intracellular space in the algal cell. In some embodiments, the cargo molecule is introduced into an organelle in the algal cell. In some embodiments, the method comprises delivering a cargo molecule into the algal cell and causing a change in cell properties of the algal cell as a result of the delivery of the cargo molecule into the algal cell.

The method may comprise steps of preparing a composition comprising the cargo molecule and a cell penetrating peptide (CPP) and exposing the algal cell to the composition. The step of preparing the composition may comprise mixing a first solution comprising the cargo molecule with a second solution comprising the CPP. The step of preparing the composition may also comprise causing the cargo molecule to associate with the CPP. The association may be covalent or non-covalent. The step of exposing the algal cell to the composition may comprise adding a third solution comprising the algal cell to the composition. The step of exposing the algal cell to the composition may comprise incubating the algal cell in the composition. Alternatively or additionally, the step of exposing the algal cell to the composition may comprise agitating the composition which contains the algal cell, the CPP and the cargo molecule in a reaction vessel. The method may comprise one or more steps of treating the algal cell to disrupt its cell wall. The treating step may comprise exposing the algal cell to an enzyme which degrades the algal cell wall. Additionally or alternatively, the treating step may comprise adding beads to the reaction vessel which contains the solution comprising the algal cell and agitating the reaction vessel to disrupt the algal cell.

The CPP used in the method facilitates algal uptake of the cargo molecule. The CPP facilitates the delivery of the cargo molecule to the algal cytoplasm, nucleus, or specific organelles such as the mitochondria and plastid. CPPs can be generally defined as short peptides containing less than 35 amino acids which are capable of transporting polar hydrophilic biomolecules across cell membranes in a receptor independent manner. CPPs can be classified into five categories: cationic peptides, peptides having hydrophobic sequences, amphipathic peptides, peptides having proline-rich and antimicrobial sequences, and chimeric or bipartite peptides [14]. CPPs within all these categories may be useful in the present method. The CPPs can be referred to as “exogenous” for the reason that they do not naturally occur within the algal host cell. The method for introducing a cargo molecule into an algal cell may comprise the steps of preparing a composition comprising a cargo molecule of interest and a CPP selected from the group consisting of cationic peptides, peptides having hydrophobic sequences, amphipathic peptides, peptides having proline-rich and antimicrobial sequences, and chimeric or bipartite peptides, and exposing the algal cell to the composition.

Any suitable CPP may be used in various embodiments. In some embodiments, the CPP may comprise a cationic CPP. A cationic CPP typically contains multiple basic amino acid residues (e.g., arginine and/or lysine). Examples of cationic CPPs include, without limitation, arginine-rich sequences [15] [16], polyarginines and peptoids [17]. For example, the CPP in the method may comprise oligoarginines of any suitable length (e.g., seven, eight, nine, ten, eleven or more arginine residues). The guanidinium head group of arginine forms bidentate hydrogen bonds with anionic groups on a cell membrane surface resulting in cell membrane translocation [18].

Examples of cationic CPPs may also include arginine-rich peptides derived from certain proteins. For example, Tat is a nuclear transcriptional activator protein that is a 101-amino acid protein required for viral replication by human immunodeficiency virus type 1 (HIV-1). The truncated fragment of HIV-1 Tat 49-57 (RKKRRQRRR) (SEQ ID NO: 1) may be used in the present method. HIV-1 Tat 49-57 (RKKRRQRRR) (SEQ ID NO: 1) is a highly basic region of HIV-1 Tat and is involved with cellular translocation [17, 19]. The CPP in the method may comprise an HIV Tat fragment or variants thereof. The CPP in the method may comprise HIV-1 Tat 49-57 (RKKRRQRRR) (SEQ ID NO: 1), or a dimer of HIV-1 Tat 49-57 (RKKRRQRRRRKKRRQRRR) (SEQ ID NO: 2), or HIV-1 Tat 48-57 (GRKKRRQRRR) (SEQ ID NO: 3). The CPP in the method may comprise Penetratin or variants thereof. Penetratin is a 16 amino acid peptide (RQIKIWFQNRRMKWKK) (SEQ ID NO: 4) corresponding to the third helix (amino acid residues 43-58) of the homeoprotein Antennapedia in Drosophila. The homeodomain structure of Antennapedia consists of three α-helices with one β-turn between helices two and three which binds DNA through a 60 amino acid sequence. Penetratin is capable of translocating through cell membranes [20, 21].

In some embodiments, hydrophobic sequences contained within signal peptides are also useful as CPPs. Signal peptides generally consist of three regions: a positively charged N-terminal region, a hydrophobic H-region, and a polar C-terminal region leading up to the signal peptidase cleavage site. The H-region is responsible for signal peptide membrane translocation [22]. Examples include the Kaposi fibroblast growth factor (FGF) signal peptide sequence and integrin β3 signal peptide sequence or variants thereof. The Kaposi fibroblast growth factor (FGF) signal peptide sequence is made up of 16 amino acids (AAVALLPAVLLALLAP) (SEQ ID NO: 5) [23]. The integrin β3 signal peptide sequence is made up of 15 amino acids (VTVLALGALAGVGVG) (SEQ ID NO: 6) [24].

Amphipathic peptides can also be useful as CPPs and possess an α-helical structure containing cationic lysine or arginine residues on one face of the helix forming hydrophobic and hydrophilic domains. Examples include MPG and Pep-1 or variants thereof. MPG is a 27 amino acid peptide (GALFLGFLGAAGSTMGAWSQPKKKRKV) (SEQ ID NO: 7) containing both a hydrophobic domain, derived from the fusion sequence of HIV gp41, and a hydrophilic domain derived from the nuclear localization sequence of SV 40 T-antigen [25]. Pep-1 is a peptide (KETWWETWWTEWSQPKKKRKV) (SEQ ID NO: 8) consisting of three domains, namely a hydrophobic domain containing multiple tryptophan residues, a hydrophilic lysine-rich domain derived from the nuclear localization sequence (NLS) of simian virus 40 (SV 40) large T antigen, and a spacer domain which improves the flexibility and the integrity of both the hydrophobic and the hydrophilic domains [26].

Proline-rich sequences and polyproline sequences may also be useful as CPPs. Proline-rich sequences and polyproline sequences tend to adopt a well-defined helical structure (polyproline II) in water, conserved even if the peptide contains only 50% proline residues. An amphipathic helix may be generated by introducing polar amino acids at certain positions. The CPP in the method may comprise the sweet arrow peptide or variants thereof. The sweet arrow peptide (SAP) (VRLPPP)3 (SEQ ID NO: 9) is derived from the proline-rich N-terminal repetitive domain of gamma-zein, a storage protein of maize [27, 28].

Antimicrobial sequences damage bacterial cell membranes during cell entry and inhibit intracellular targets resulting in their microbiocidal properties. Dermaseptins constitute a large family of polycationic antimicrobial peptides which are expressed in the skin of certain tree frogs. S413-PV (ALWKTLLKKVLKAPKKKRKVC) (SEQ ID NO: 10) is an antimicrobial peptide composed of 13 amino acids derived from the dermaseptin S4 peptide and the SV40 large T-antigen NLS [29].

In some embodiments, the CPP may comprise a chimeric or bipartite peptide containing two or more of the peptide motifs described above herein. Transportan is a 27 amino acid chimeric peptide (GWTLNSAGYLLGKINLKALAALAKKIL) (SEQ ID NO: 11) derived from 12 residues of the N-terminal part of the neuropeptide galanin linked to the 14 amino acids of the wasp venom mastoparan [30]. pVEC is an 18-amino acid-long peptide (LLIILRRRIRKQAHAHSK) (SEQ ID NO: 12) derived from the murine sequence of the cell adhesion molecule vascular endothelial cadherin (amino acid 615-632) [31]. Human calcitonin (hCT) is a 32 amino acid peptide hormone involved in the regulation of calcium homeostasis. Attachment of the SV40 large T-antigen NLS to the side chain of Lys 18 in hCT9-32 may result in a branched peptide hCT9-32-br (LGTYTQDFNK*FHTFPQTAIGVGAP) (-AFGVGPDEVKRKKKP) attached to K* [32]. Mouse prion protein mPrPp (MANLGYWLLALFVTMWTDVGLCKKRPKP) (SEQ ID NO: 13) is composed of the N-terminal PrP sequence which is a hydrophobic signal peptide followed by a basic putative NLS of six residues [33].

In some particular embodiments, the CPP used in the method is selected from the group consisting of Tat 49-57 (GRKKRRQRRR) (SEQ ID NO: 1), Tat 48-57 (GRKKRRQRRR) (SEQ ID NO: 3), a polyarginine, Transportan (GWTLNSAGYLLGKINLKALAALAKKIL) (SEQ ID NO: 11), and Penetratin (RQIKIWFQNRRMKWKK) (SEQ ID NO: 4). The polyarginine may comprise

(Arg)7 (RRRRRRR) (SEQ ID NO: 14), (Arg)8 (RRRRRRRR) (SEQ ID NO: 15), (Arg)9 (RRRRRRRRR) (SEQ ID NO: 16), (Arg)10 (RRRRRRRRRR) (SEQ ID NO: 17), or (Arg)11 (RRRRRRRRRRR) (SEQ ID NO: 18).

The cargo molecule chosen for delivery into the algal host cell may be selected from among all useful biomolecules, including polynucleotides of either the DNA or RNA type such as oligonucleotides, intact genes, intact expression cassettes, plasmids, sRNA, siRNA in both sense and anti-sense orientations, PNA, polypeptides including oligopeptides, lipids including liposomes, carbohydrates and the like. In some embodiments, the cargo molecule may comprise drugs, or imaging agents. All of these cargo molecules may be delivered into an algal host cell with the assistance of a CPP. Because the cargo molecule does not occur naturally within the algal host cell, it is referred to herein as being “heterologous”.

In some embodiments, the method comprises preparing a composition comprising a nucleic acid molecule and a cell penetrating peptide (CPP), exposing the algal cell to the composition, and introducing the nucleic acid molecule into the algal cell to transform the algal cell, wherein the CPP facilitates the uptake of the nucleic acid molecule by the algal cell.

In some embodiments, the cargo molecule is a marker molecule that allows selection of the algal transformant. These markers can be enzymes or genes or other biomolecules that confer a survival advantage to the algal transformant, and allow for its selection when grown under conditions requiring the marker for survival. In the alternative, the marker can be any biomolecule that is detectable in the algal transformant, such as green fluorescence protein (GFP) or a gene encoding it, which allows the transformant to be detected. Still other markers and reporter genes that can be introduced into algae cells using CPPs include the following:
aadA Adenylyl transferase (resistance to spectinomycin)
als Acetolactate synthase (resistance to sulfonylurea herbicides
aph VIII Aminoglycoside 3′phosphotransferase (resistance to paramomycin)
ars Arylsulphatase
ble Bleomycin binding protein (resistance to zeocin)
cat Chloramphenicol acetytransferase (resistance to chloramphenicol)
cryl-1 Ribosomal protein S14
£-frustulin Calcium binding glycoprotein
gjjJ′ Modified green fluorescent protein
gluti Glucose transporter
gus p-glucoronidase
hpt Hygromycin B phosphotransferase
hupl Hexose transporter
luc Luciferase
nat Nourseothricin resistance
npt11 Neomycin phosphotransferase 11 (resistance to G418)
oee-1 Oxygen evolving enhancer protein
sat-1 Nourseothricin resistance.

In addition to markers and reporter genes, the cargo molecule can be a nucleic acid molecule that comprises a gene of interest which encodes a protein of interest. These include genes that encode proteins useful to engineer the genetics of the algae, such as for enhanced survival, reduced proteolytic enzyme production, and the like. Particular genes of interest are those that encode proteins involved in fatty acid metabolism, carbohydrate metabolism, genes associated with stress tolerance in growth conditions that involve altered pH, altered salinity or altered temperature. Particular genes of interest as cargo molecules include Bcl-xL (an abbreviation for B-cell lymphoma extra-large) which is a known inhibitor of apoptosis; genes involved in ethanol production including pyruvate decarboxylase (PDC) and alcohol dehydrogenase (ADH); genes involved in methanol production including formate dehydrogenase (FateDH), formaldehyde dehydrogenase (FaldDH) and/orADH; and genes involved in the production of butanol including pyruvate-ferredoxin oxidoreductase, acetyl-CoA-acetyl transferase, hydroxybutyryl-CoA dehydrogenase, crotonase, butyryl CoA dehydrogenase, phosphobutyrylase, and butyrate kinase. Also useful as cargo molecules or genes of interest are the FatB genes such as Arabidopsis thaliana FATB NM_{—}100724, California Bay Tree thioesterase M94159, Cuphea hookeriana 8:0- and 10:0-ACP specific thioesterase (FatB2) U39834, Cinnamomum camphora acyl-ACP thioesterase U31813, Diiploknema butyracca chloroplast palmitoyl/oleoyl specific acyl-acyl carrier protein thioesterase (FatB) AY835984, as well as acetyl CoA synthetase genes such as Arabidopsis ACS9 gene GI: 20805879; Brassica napus ACS gene GI: 12049721; oryza sativa ACS gene GI: 115487538, and the like.

To drive expression of the gene of interest within the algal cell, in some embodiments the nucleic acid molecule introduced into the algal cell comprises a promoter and/or a control sequence which are linked operably to the gene of interest. The promoter should be a promoter that is functional in the algal host cell. Promoters from algae species may be used. In some embodiments, promoters from plant species may be used. In some embodiments, promoters from virus or bacterial sources may be used. For example, suitable promoters may include the promoters of the following genes: nlal (Nitrate reductase from C. reinhardtii,), rbcS2 (Rubisco small subunit from C. reinhardtii), hspZOA (Heat shock protein from C. reinhardtii), psaD (Photosystem 1 complex protein from C. reinhardtii), cop (Chlamyopsin from C. reinhardii), nos (Nopaline synthase from A. tumefaciens), CaA4V/35S (Cauliflower mosaic virus 35S), ubil-Q (ubiquitin-Q from Zea mais) SWO (simian virus 40), ab/7-W (ubiquitin-Q from Zea mais), fcp (Fucoxanthin chlorophyll-a or -c binding protein), nos (Nopallne synthase from A. tumefaciens), p7′2′(from A. tumefaciens).

When necessary for efficient expression of the gene of interest, the nucleic acid molecule comprising the promoter and the gene of interest may further comprise, linked operably to the 3′ end of the gene, a 3′-untranslated region for efficient termination of expression. These regions may comprise transcription and translation termination sequences and poly-adenylation sequences. The 3′-nontranslated regions may be obtained from the flanking regions of genes from algae, yeast, bacteria, plant or other eukaryotes. Some examples of useful 3′-untranslated regions include those associated with genes such as the pea ribulose biphosphate carboxylase small subunit E9 gene, the the noplaine synthase gene and the SV 40 gene, among many others.

To introduce the cargo molecule into the algal host cell, the CPP and the cargo molecule may be mixed to yield particles in which the cargo molecule becomes nucleated by the CPP through a physical, non-covalent interaction. Alternatively, the CPP and cargo molecule may be chemically coupled to yield covalently coupled hybrid molecules.

To generate non-covalently associated CPP/cargo particles, the CPP may be provided in stoichiometric excess relative to the cargo molecule (e.g., 1.2:1, 1.5:1, 2:1, 3:1, 4;1, 5:1, 8:1, 10:1, 20:1 or more in molar ratio), so that a number of CPP molecules are available for interaction with a given molecule of cargo. Conditions of mixing are chosen so that the final volume is small (e.g., less than or equal to 200 μl, or 150 μl, or 100 μl, or 50 μl, or 20 μl) and the liquid matrix contains a minimum of metal ions or chelating agents. In addition, the CPP can be selected with reference to the physical properties of the cargo molecule: a cargo molecule that is charged abundantly with negative charges will typically be associated with a CPP that has correspondingly numerous positive charges. For example, in some embodiments where the cargo molecule is a polynucleotide (e.g., a DNA molecule), the CPP is chosen from Tat (49-57), (Arg)9, Transportan, or Penetratin.

Covalent coupling of the cargo molecule and the CPP may be achieved using a suitable chemical method. A common and simple way of chemically conjugating nucleic acids to peptides is to link through the thiol group of a cysteine residue. This can be achieved in essentially two ways depending on the modification carried out on the 3′- or 5′-end of the nucleic acid strand. Commercially available nucleic acids which have been chemically modified at the 3′- or 5′-end with a thiol group represents the most straightforward approach. Oxidative coupling between the thiol modified nucleic acid and the thiol group of a cysteine residue in the CPP can be achieved for example by incubating a mixture comprising the thiol modified nucleic acid and the cysteine-containing CPP for 1 h at 40° C. with a thiol cross-linking agent (e.g., diamide from Sigma). In some embodiments, this procedure is used to couple siRNA to Transportan or Penetratin. For example, the Transportan or Penetratin may be synthesized with a cysteine residue on one end of the peptide, and then conjugated to a chemically treated siRNA.

Another approach is to activate one of the thiol containing components with a pyridylsulfide before addition of the second thiol component. This allows specific heterodisulfide formation without concomitant homodisulfide formation of the peptide or nucleic acid component. The reaction may be carried out in phosphate buffered saline (PBS) and is very rapid. The above coupling chemistry can also be carried out with 3′- or 5′-amino modified primary amine group at the 3′- or 5′ is reacted with an active N-succinimidyl-3-(2-pyridyldithio) propionate (group a hetero-functional coupling reagent SPDP), to produce 2-pyridyl disulfide activated nucleic acids which then reacts with the thiol group present in the peptide. For example, SPDP may be used to couple a cargo molecule to a lysine residue of a CPP such as Penetratin.

Another approach uses suitably modified 3′- or 5′-nucleic acids, with a terminal carboxylic acid group which could be activated using carbodiimide/N-hydroxy succinimide chemistry to give an ‘activated ester’. Coupling of this ‘activated’ nucleic acid to the amine groups of lysines (forming a peptide bond) may be achieved in PBS by stirring the two components at room temperature for 1 hour.

In an alternative approach the 3′- or 5′-amine terminated nucleic acids may be converted into a carboxylic acid by reacting with glutaric anhydride. The amine group will ring open glutaric anhydride at room temperature, forming an amide linkage and liberating a carboxylic acid. This nucleic acid is now terminated with a carboxylic acid group which can be converted into an ‘active ester’ and coupled to a lysine residue of the CPP.

In some embodiments, the cargo molecule of interest (e.g., DNA) is simply mixed at room temperature with a solution comprising the selected CPP, and the combination then is allowed to associate for 10 minutes. Limiting the incubation time to 10 minutes ensures that CPP-DNA aggregates don't become too large and precipitate out of solution. In one embodiment, the room temperature solution in which the CPP and DNA are dissolved in is ddH₂O. In some embodiments, the final volume of the reaction is less than or equal to 100 μl. This ensures that the algae media is made slightly hypotonic which will enhance the uptake of the CPP-cargo complex into the algae cells. Thereafter, the selected algal host cell may be introduced to the CPP-cargo mixture.

Before or after the introduction of algal cells into the CPP-cargo mixture, algal cells of a species comprising an outer cell wall may be treated to disrupt the cell wall, such as by agitation in the presence of glass beads. The glass beads may have a diameter in the range of 0.4-0.5 mm. An additional or alternative method for cell wall disruption involves the use of autolysin for 30-60 min, or the use of some other cell-wall degrading enzyme as described further below herein. After agitation, the glass beads are then allowed to settle in the reaction vessel.

After incubation in the CPP-cargo mixture for a period that can be about 1 hour or more or some other suitable time period, the algae cells may be plated on a solid culture media to select for transformants. When the CPP-cargo particles are prepared, they can be introduced into an algal culture in a culture medium that allows for their selection, the conditions for which are determined by the type of marker introduced as the cargo molecule.

The present method can be applied to any species of algae, including marine algae and freshwater algae, and including green algae, red algae, brown algae, diatoms, and euglenids, and including chlorophyll-C containing algae, and including unicellular algae and multi-cellular algae. Examples of suitable algal hosts include: C. reinhardtii, Chiarella ellipsoidea, Chiarella saccharophila, C. vulgaris, Haematococcus pluvialis, V. carteri, Chiarella sorokiniana, Chiarella kessleri, Ulva lactuca, Dunaliella viridis, D. salina, Nannochloropsis oculata, T. pseudonana, P. tricornutum, Navicula saprophila, Cylindrotheca fusiformis, Cyclotella cryptica, Thalassiosira weissflogii, Laminariajaponica, Undaria pinnatifada, Rhodophytes, C. merolae, Porphyra yezoensis, Porphyra miniata, Kappaphycus alvarezii, Gracilaria changii, Porphyridium sp., Amphidinium sp., Symbiodinium microadriaticum, Euglena gracilis, Auxenochlorella protothecoides, Scenedesmus obliquus, Dunaliella tertiolecta, Botryococcus braunii, Spirulina palatensis, Botryococcus braunii, Chiarella sp., and Chiarella protothecoides. In some particular embodiments, the present method is applied to generate algal transformants of a species selected from Chlamydomonas reinhardtii, Spirulina palatensis, Botryococcus braunii, Chiarella sp., and Chiarella protothecoides.

In some embodiments, the CPP may remain in the transformed algal cell as a residual molecule. In other embodiments, the CPP may be degraded by intracellular enzymes in the transformed algal cells. The transformed algae may be useful for a variety of commercial purposes. In general, the algal transformants are cultured under conditions suitable for their growth, and the product of the gene of interest, or the algal biomass itself, is recovered from the culturing medium. For increased biomass production, the algal culture can be scaled up to for example between about 1 L to 10,000 L or more of culture.

The methods described herein have many advantages. They do not require expensive or specialized equipment. They are relatively easy to perform. They do not cause significant damage to the algal cells.

In one aspect of the invention, there is provided a composition for introducing a cargo molecule into an algal cell, the composition comprising a cargo molecule and a cell penetrating peptide. In one aspect of the invention, there is provided a composition for transforming an algal cell, the composition comprising a nucleic acid molecule and a cell penetrating peptide.

In one aspect of the invention, there is provided a transformed algal cell produced according to the methods described herein. The transformed cell may have an altered cell property as a result of the transformation. In some embodiments, the transformed cell comprises a heterologous gene integrated in its genome. In some embodiments, the transformed cell expresses a protein encoded by the heterologous gene.

Further aspects of the invention and various example embodiments of the invention are described below.

### EXAMPLES

The following examples are provided to illustrate embodiments of the present invention but they are by no means intended to limits its scope.

### Example 1

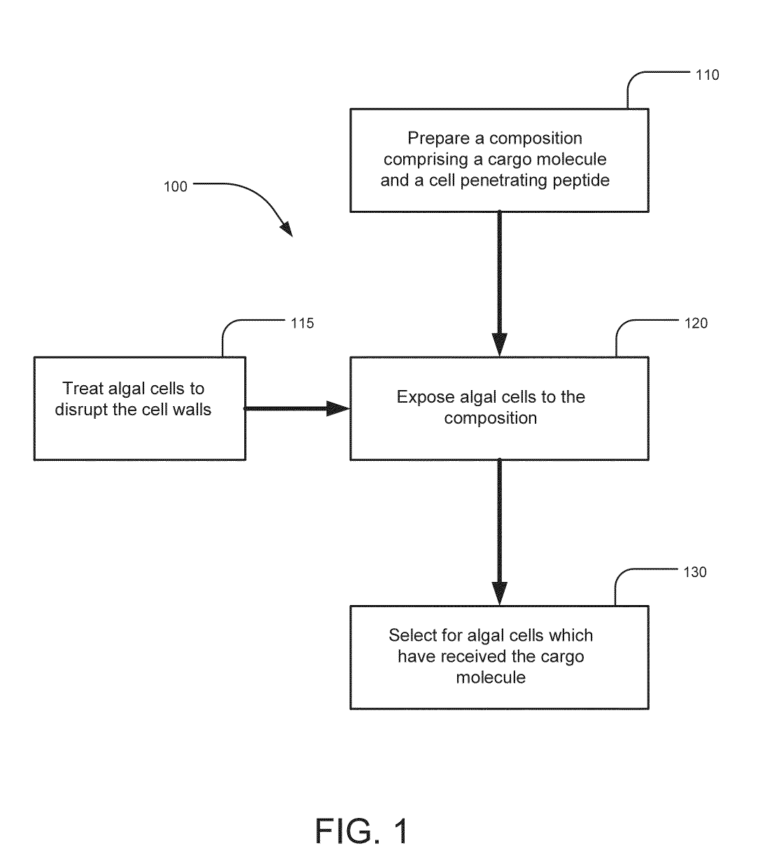

FIG. 1 schematically illustrates a method 100 for introducing a cargo molecule into an algal cell according an example embodiment of the invention. In the illustrated embodiment, method 100 comprises step 110 of preparing a composition comprising a cargo molecule and a cell penetrating peptide; and step 120 of exposing the algal cells to the composition for a period of time. Method 100 may also optionally comprise step 115 of treating the algal cells to disrupt the cell walls of the algal cells. After step 120, the method 100 comprises step 130 of selecting for algal cells which have received the cargo molecule. In method 100, the cargo molecule may be polynucleotides, polypeptides including oligopeptides, lipids including liposomes, carbohydrates, therapeutic drugs, or imaging agents. Step 115 may comprise treating the algal cells with an enzyme which degrades the algal cell walls. Step 115 may further comprise adding beads to the composition containing the algal cells and agitating the composition. In some embodiments, the agitating step may comprise vortexing the composition for a period of time, e.g., in the range of 10 s to 1 min. In some embodiments, beads are added to the composition containing the algal cells, the CPP, and the cargo molecule, and the composition is then agitated. Agitating the beads, the algal cells, the CPP, and the cargo molecule all together in a single reaction vessel may improve the uptake of the cargo molecule by the algal cells.

### Example 2

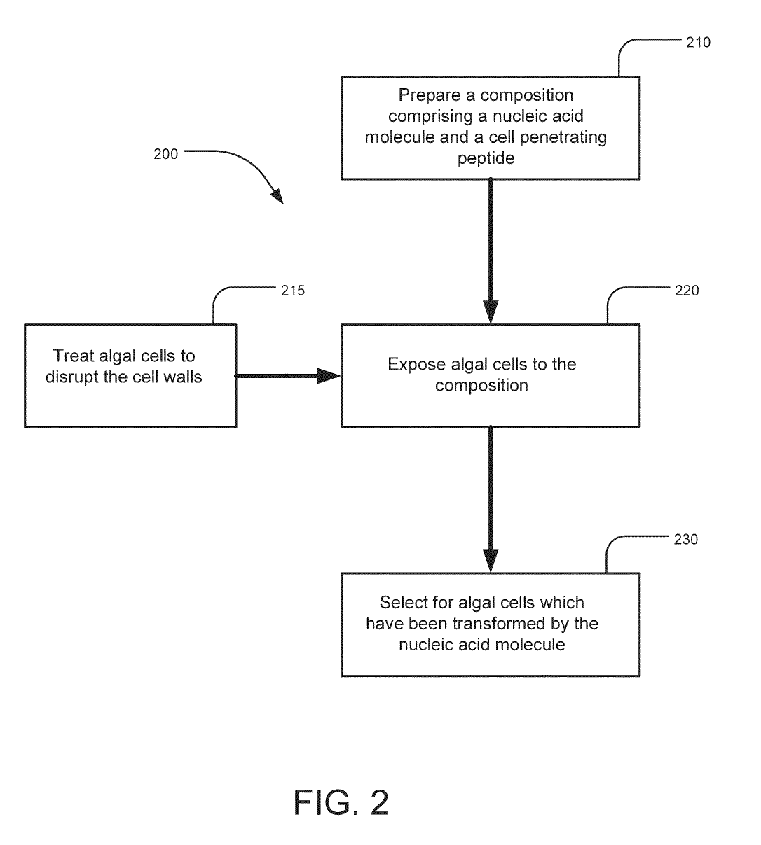

FIG. 2 schematically illustrates a method 200 for transforming an algal cell according an example embodiment of the invention. In the illustrated embodiment, method 200 comprises step 210 of preparing a composition comprising a nucleic acid molecule and a cell penetrating peptide; and step 220 of exposing the algal cells to the composition for a period of time. Method 200 also comprises step 215 of treating the algal cells to disrupt the cell walls of the algal cell. After step 220, the method 200 comprises step 230 of selecting for algal cells which have been transformed by the nucleic acid molecule. In method 200, the nucleic acid molecule may comprise a plasmid. The plasmid may be circular, or linearized (e.g., linearized with a restriction enzyme). The plasmid may comprise a detectable marker, a selectable marker, or a reporter gene. The plasmid may comprise a gene of interest which encodes a protein of interest. In some embodiments, the plasmid may comprise a promoter which is operably linked to the gene of interest for driving the expression of the gene of interest in the algal cell. In some embodiments, the plasmid may comprise one or more DNA sequences which facilitate the integration of the gene of interest into the genome of the algal cell through homologous recombination or other means.

### Example 3

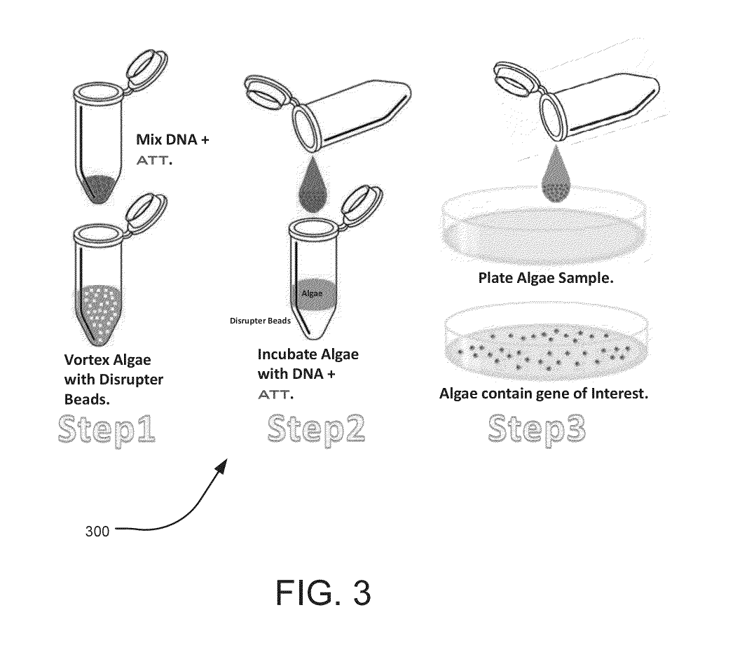

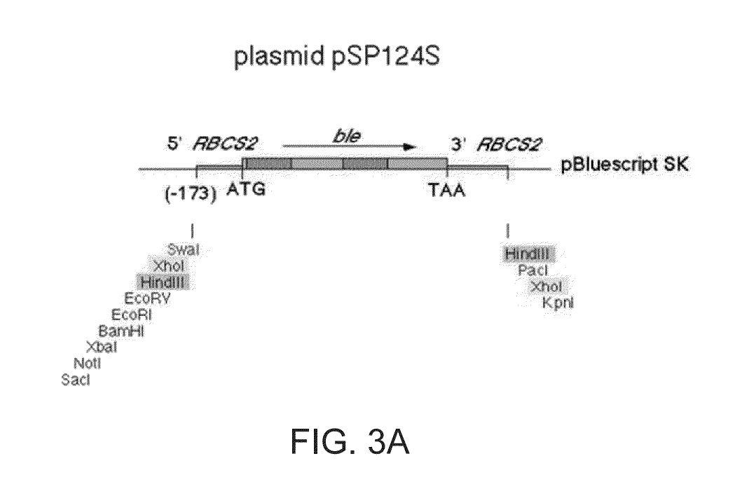

FIG. 3 graphically illustrates an example protocol 300 for transforming an algal cell according an example embodiment of the invention. In step 1 of protocol 300, a plasmid containing a gene of interest and a selectable marker to a final concentration of 20 ng/μl is provided. Then 50 μl of the plasmid DNA is added to a selected CPP in a 1.5 ml microcentrifuge tube and incubate for 10 min at room temperature. During this incubation 0.4 ml of an algae culture (1-2×10⁶ cells per ml) is added to a 1.5 ml microcentrifuge tube containing 300 mg of disrupter beads (0.4-0.5 mm glass beads). The tube is agitated at top speed for 15-30 seconds using a vortex. In FIG. 3, the CPP is referred to as “ATT”. In step 2 of protocol 300, the disrupter beads are allowed to settle to the bottom of the tube and 50 μl of the CPP-DNA composition is added to the algae cells. Cells are incubated for 1 hour. In step 3 of protocol 300, algae cells are plated on a solid media containing an appropriate amount of selective agent. Algae colonies that grow will contain the gene of interest.

### Example 4

Experiments were conducted to optimize algal growth conditions, measuring algal growth curves to determine the logarithmic phase of cell multiplication. Sensitivity tests were conducted to validate the selection methods for selecting transformed algal cells after the genomic insertion of a marker gene was achieved using the transformation methods as described herein. Preliminary sensitivity tests were conducted to check the viability of algae cells after certain treatments to be used in the transformation experiments. Four different cell penetrating peptides (CPPs) were tested, namely,

(SEQ ID NO: 11)
ATT1 = Transportan (GWTLNSAGYLLGKINLKALAALAKKIL)
(SEQ ID NO: 3)
ATT2 = Tat (48-57) (GRKKRRQRRR)
(SEQ ID NO: 4)
ATT3 = Penetratin (RQIKIWFQNRRMKWKK)
(SEQ ID NO: 16)
ATT4 = (Arg)9 (RRRRRRRRR)

For convenience and simplicity, these four CPPs are termed ATT1, ATT2, ATT3 and ATT4 respectively. These CPPs may be made using peptide synthesis methods. Peptides were obtained from AnaSpec.

Experiments were conducted using agitation of algal cells with glass beads in combination with the use of enzymatic cell wall destruction treatment. The inventor has observed that the use of enzymatic cell wall destruction treatment enhances algal transformation rates.

Transformation experiments were conducted on C. reinhardtii Nit1, cw-15. The inventor has utilized a plasmid pSP124S which contains the Bleomycin (alternate name Zeocin™) resistance gene. The ble gene originates from the tallysomycin-producing actinomycetes species Streptoalloteichus hindustanus and encodes a small (13.7 kDa) protein conferring resistance to tallysomycin and related antibiotics including bleomycin, phleomycin and zeomycin. These glycopeptide antibiotics act by breaking down DNA: the BLE protein prevents this by binding the antibiotics with strong affinity. The gene has been developed as a dominant selectable marker for both prokaryotes and lower and higher eukaryotes. To develop a ble marker for Chlamydomonas nuclear transformation, the ble coding region was fused to the 5′ and 3′ regulatory regions of the Chlamydomonas RBCS2 gene to create a marker (plasmid pSP108) that allows direct selection for phleomycin-resistant transformants. This was reported in Stevens, D. R., Rochaix, J.-D. and Purton, S. (1996). “The bacterial phleomycin resistance gene ble as a dominant selectable marker in Chlamydomonas.” Mol. Gen. Genet. 251, 23-30. However, the transformation efficiency using this marker was low. This construct was therefore improved this by introducing Chlamydomonas intronic sequences into the ble gene and shortening the 5′ RBCS2 region. These modifications are described in Lumbreras, V., Stevens, D. R. and Purton, S. (1998) Plant J. 14, 441-447, which is incorporated by reference herein. The best version of the construct (pSP124) described in the paper then underwent some additional minor modifications to give pSP124S as described below.

As shown in FIG. 3A, the marker of pSP124S comprises: i) the RBCS2 promoter region extending from the −173 position (relative to the transcription start) to the translation start; ii) the ble coding region, into which has been inserted two copies of RBCS2 intron 1 [one intron immediately downstream of the ATG, one in the middle of the coding region]; iii) a 231 by fragment containing the 3′ untranslated region of RBCS2, including the putative polyadenylation signal. The cassette is 1.2 kb in size (can be excised using XhoI or HindIII) and is cloned into pBluescript SK-. Several rare RE sites (PacI, SwaI, etc.) have been introduced at the ends of the marker to facilitate DNA analysis of transformants and the rescue of genomic flanking sequence. The pSP124S plasmid was obtained from the Chlamydomonas Center (http://www.chlamy.org/chlamydb.html).

The inventor has tested both circular plasmid and linearized plasmid in transformation experiments. In some experiments, the inventor has observed that the use of linearized plasmid, as opposed to circular plasmid, provides a better transformation rate. The inventor obtained algal transformants using each one of the four peptides (ATT1 to ATT4). In some experiments, the inventor observed that the transformation rate was higher using ATT1, as opposed to the other three peptides. In some experiments, the inventor estimated that the transformation efficiency is in the range of 1% to 3% (for example, about 2%).

### Strains and Cell Cultures

After a successful cell multiplication, stock algae cell cultures were grown for 3-4 generations prior to the inoculation of experimental cultures. To ensure the maintenance of axenic algae, cultures were grown in media supplemented with a low concentration of antibiotics.

Cells of C. reinhardtii (Nit1-305,cw-15) were used. These cell lack nitrate reductase gene function and therefore require a reduced form of NH₄ for growth. The cells were inoculated into ½R medium which is the modified version of SGII-NH₄ medium (Kindle, 1990) replacing KNO₃ with NH₄NO₃ from Sanger-Granick medium II (Sanger and Granich, 1953). When cell growth reached approximately 2-3×10⁶ cells per ml, the cells were inoculated into SGII-NH₄NO₃ medium for 3-4 generations. All the culture maintenance and experimental growth cultures were grown in an incubator with 16 h:8 h as day:night cycles. Temperature was maintained at 24±1° C.

### Determination of Growth Curves and Sensitivity Tests

Fresh cell cultures of C. reinhardtii (Nit1-305,cw-15) in SGII-NH₄ medium over a period of 3-4 generations under the aforementioned conditions optimal for growth of each strain. Aseptic conditions were strictly practiced to maintain continuous purity of the cells.

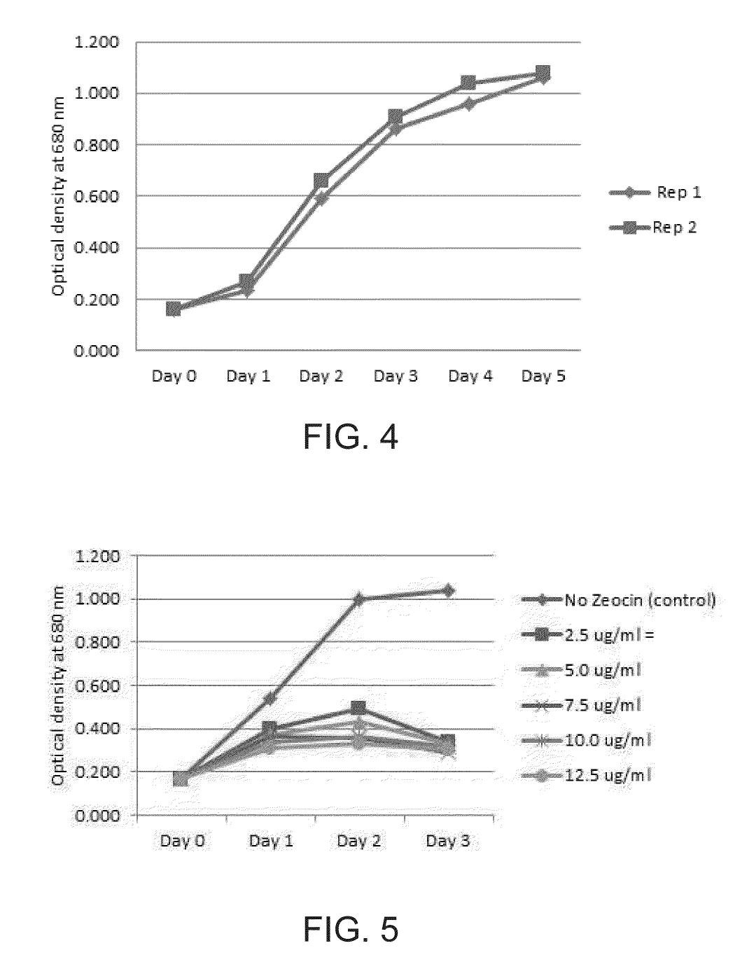

The cells of the algal strains were inoculated into fresh growth media suitable for the cell types and the cultures were placed to grow under optimal conditions (see strains and cell cultures section above herein). Measurements of optical density (OD) and/or cell counts for determination of cell density of the cultures were performed at regular interval of time (24 h) until the cell growth reached to stationary phase. The OD values were measured at 680 nm wavelength using a spectrophotometer (Milton Roy, SPECTRONIC 20D). Cells were counted using a disposable hemocytometer C-Chip (DHC-N01) (INCYTO Co., Ltd.) using a light microscope (OLYMPUS, Japan). Growth curves were obtained by plotting cell density values (ODs) against time period in days (see Table 1 and FIG. 4).

TABLE 1
Growth curve based on the values of OD (at 680 nm) of Chlorella spp.
(wild-type) and Chlaymydomonas reinhardtii (Nit1-305, cw-15)
mutant in TAP and SGII-NH₄NO₃ media, respectively.

| | Chlaymydomonas reinhardtii | |
|---|---|---|
| Time in days | Rep 1 | Rep 2 |
| Day 0 | 0.161 | 0.16 |
| Day 1 | 0.235 | 0.265 |
| Day 2 | 0.590 | 0.66 |
| Day 3 | 0.865 | 0.91 |
| Day 4 | 0.960 | 1.04 |
| Day 5 | 1.060 | 1.08 |

FIG. 4 shows growth curve analysis of C.reinhardtii based on OD values at 680 nm showing logarithmic phases: two replicates of C.reinhardtii culture were grown.

Minimal inhibitory concentrations (MIC) Zeocin™. Liquid cultures were inoculated to ˜10⁴ cells/ml and ˜10⁶ cells were spread on the solid media plates. Every day, OD values were measured for liquid cultures and the plates were inspected to record growth of cells. The MIC was decided at which the growth was inhibited in both the liquid cultures (see FIG. 5) and on the agar plates. FIG. 5 shows graphs showing determination of minimal inhibitory concentration of Zeocin™ for C. reinhardtii. The plots are based on growth response to different concentrations of the antibiotic as OD values at 680 nm.

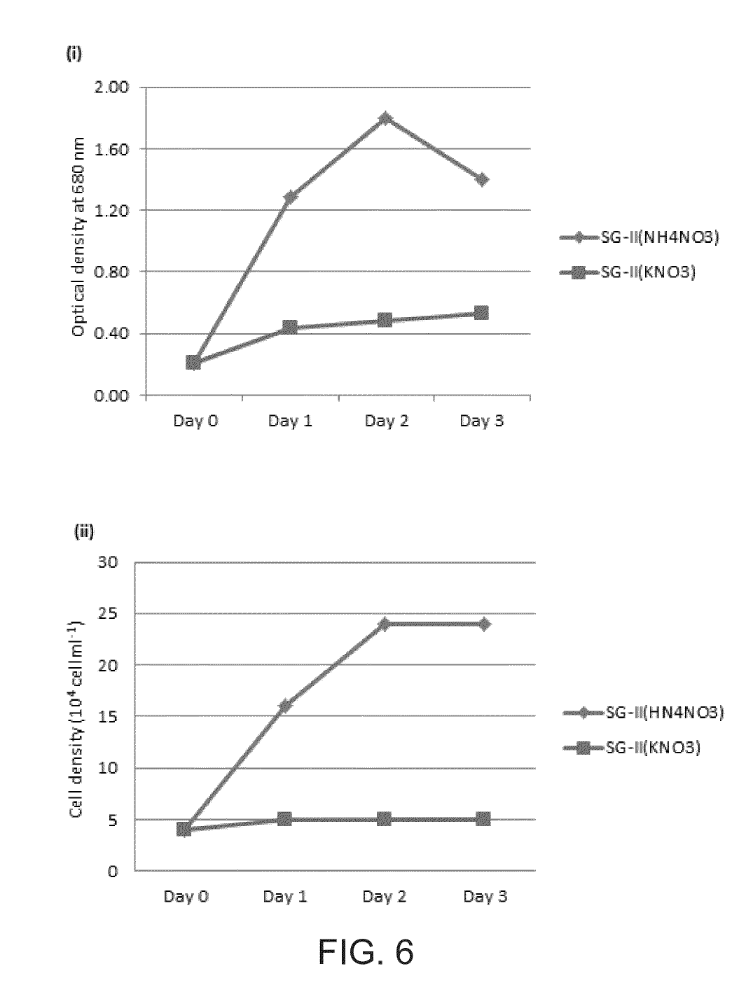

A sensitivity test of C. reinhardtii (Nit1-305, cw-15) mutant for SGII-NH₄ medium and SGII-NO₃ medium was performed. Cells from stock culture grown in SGII-NH₄ medium were spun, washed twice with sterile distilled water to remove NH₄ traces. The washed cells were then used as inoculums for the sensitivity test against KNO₃, the selection agent, for transformants containing inserted Nitrate reductase gene which is mutated in C. reinhardtii (Nit1-305,cw-15). The culture in both media were inoculated to 4×10⁴ cells/ml. Cell densities were determined by measuring OD values and cell counting daily (see Table 2 and FIG. 6). Agar plates were also prepared for both media types and the growth pattern or cell bleaching were inspected daily.

TABLE 2
A sensitivity test comparing growth of non-transformed
C. reinhardtii (Nit1-305, cw-15) mutant in SGII-NO₃ medium
in comparison to growth in SGII-HN₄NO₃ medium as control.
The response is measured in terms of ODs at 680 nm.

| | Chlamy Nit1, | | Chlamy Nit1, | |
|---|---|---|---|---|
| | cw-15 SG-II(NH4NO3) | | cw-15 SG-II(KNO3) | |
| Time in Days | OD at 680 nm | Cell # | OD at 680 nm | Cell # |
| Day 0 | 0.210 | 4 × 10\hat{ }4 | 0.212 | 4 × 10\hat{ }4 |
| Day 1 | 1.290 | 16 × 10\hat{ }4 | 0.434 | 5 × 10\hat{ }4 |
| Day 2 | 1.800 | 24 × 10\hat{ }4 | 0.486 | 5 × 10\hat{ }4 |
| Day 3 | 1.400 | 24 × 10\hat{ }4 | 0.526 | 5 × 10\hat{ }4 |

FIG. 6 is a schematic presentation of difference in growth curves due to sensitivity of non-transformed C. reinhardtii (Nit1-305,cw-15) mutant in SGII-(KNO₃) medium in comparison to the growth in SG-II(NH₄NO₃) medium. The plots are based on (i) OD values at 680 nm and (ii) cell count.

### Transformation Experiments

Transformation experiments were conducted. Some steps are modified from the glass bead method of Kindle (1990) and Lumbreras et al. (1998). Cell culture was grown in 900 ml of SGII+NH₄NO₃ medium, divided in 3 aliquots into 500 ml Erlenmeyer flasks each containing 300 ml of the culture. The culture was grown for two days until the cell growth reached mid-log phase and cell density reached to 1×10⁶ cells ml⁻¹ (OD at 680 nm=0.6-0.8). Cells were collected at 3000 rpm for 10 minutes to obtain 9×10⁸ cells in total. In all transformation experiments for Chlamydomonas reinhardtii, SGII+NH₄NO₃ medium was used in downstream experimental steps. During the transformation experiment using CPPs to deliver pMN24 (a plasmid containing nitrate reductase gene, see Fernandez E et al., 1989,Proc. Natl. Acad. Sci. USA 86:6449-6453 which is incorporated by reference herein) selection medium (SGII-KNO₃) was used. Cells were washed after grown in SGII-NH₄NO₃ medium to remove NH₄ traces. Cells were divided into 3 aliquots each of 3×10⁸ cells. Two aliquots of the harvested cells were treated with cell wall degrading enzymes one with cellulase from Aspergillus niger (Sigma, Canada) and the other with lysozyme from chicken egg white (Sigma, Canada).

Lysozyme treatment: Lysozyme from chicken white egg was purchased (Sigma-Aldrich, Canada). Enzymatic assay protocol was modified from Sigma-Aldrich product information publication. For cell lysis/cell wall disruption, use a freshly prepared lysozyme solution (10 mg/ml) in sterilized 10 mM Tris-HCL, pH 8.0. Add 25 μl of a freshly prepared lysozyme to the required number (−5.0×10⁷) of cells/transformation reaction, mix by vortexing for 3 seconds. Incubate the lysis mixture for 15 minutes at 37° C. (based on the viability test on algae strain, data not shown). Centrifuge the cells and discard the supernatant containing lysozyme. To stop the reaction, wash the cells twice with media and collect the cells at 3000 rpm for 10 minutes. Proceed to downstream applications and/or transformation experiment.

Cellulase treatment: Cellulase (EC 3.2.1.4) was purchased from Sigma-Aldrich, Canada. Enzymatic assay protocol was modified from Sigma-Aldrich product information publication. Just before use, prepare solution containing 8 units/ml (26.7 mg/ml) of cellulase in sterilized cold distilled water. Add 500 μl of a freshly prepared cellulase solution to the required number (−5.0×10⁷) of cells, immediately mix by swirling. Incubate the lysis mixture for 15 minutes at 37° C. (based on the viability test on algae strain). Centrifuge the cells and discard the supernatant containing lysozyme. To stop the reaction, wash the cells twice and collect the cells at 3000 rpm for 10 minutes. Proceed to downstream applications and/or transformation experiment.

The third aliquot was not treated with any enzyme.

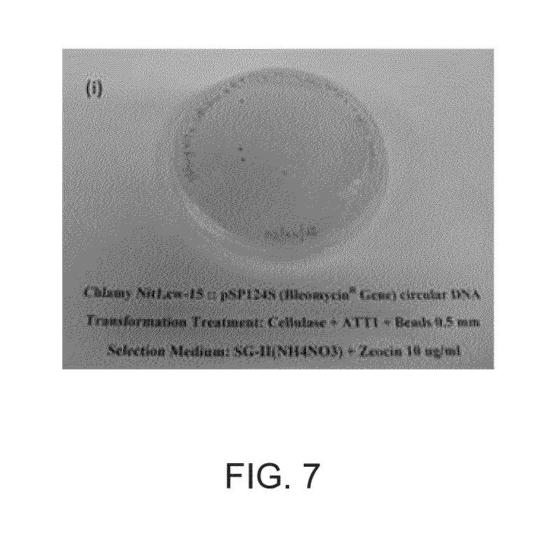

After enzymatic treatments, the cells were washed twice with the medium to stop the reaction. Cells of each aliquot were re-suspended in fresh medium (cell concentration=3×10⁸ml⁻¹) and used 300 μl of cell suspension (−5×10⁷ number of cell) in each transformation reaction. Cells were transferred to 15 ml Falcon tube containing sterilized 300 mg glass beads of 0.5 mm size. Just prior to starting the next step, 1 μg plasmid DNA and one of the four ATT(s) tested were mixed. One of the four ATT-plasmid mixtures were added to the algal cells and beads suspended in media. The cells, ATT and DNA mixture and the beads were vortexed for 15 seconds. 10 ml of medium was added to the transformation reaction, and the tubes were incubated overnight with shaking at 100 rpm in incubator to allow cell recovery and expression of the gene. The cell suspensions were transferred to fresh tubes to remove beads and cells were spun by centrifugation for 5 minutes at 3000 rpm. The cells were re-suspended in 2 ml of 0.5% agar medium by gentle pipetting and were plated on 2% agar medium supplemented with 10 μg ml⁻¹ Zeocin. Agar was allowed to set and the plates were inverted after sealing with parafilm. Plates cultures were grown in an incubator with 16 h:8 h as day:night cycles, temperature was maintained at 24±1° C. Transformed colonies become visible after 14 days (see FIGS. 7-9).

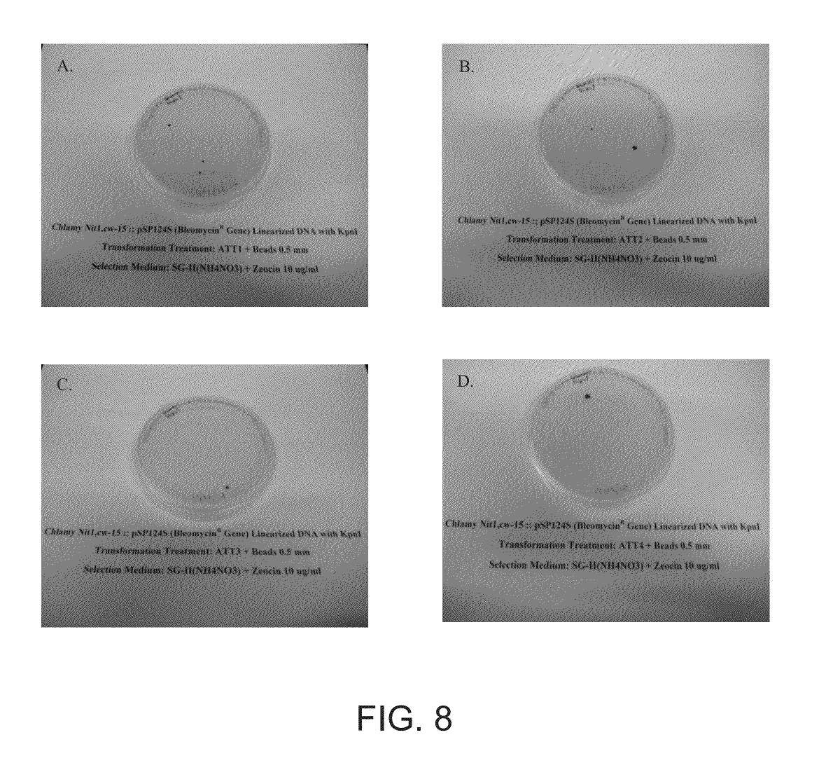

FIGS. 7-9 are example pictures of successful transformation showing transformed colonies on selection plates. Each picture contains text which describes the algae type, plasmid name with gene insert and the transformation treatment. FIG. 7 shows C. reinhardtii (Nit1-305,cw-15) transformed with circular pSP124S plasmid DNA (Bleomycin^{R} Gene) in the presence of ATT1. In this experiment, the cell walls of the cells were treated with cellulase and bead disruption using beads of 0.5 mm size. The selection medium used is SGII-NH₄NO₃ medium containing Zeocin at 10 μg/ml. FIG. 8(A) shows C. reinhardtii (Nit1-305,cw-15) transformed with pSP124S plasmid DNA linearized with KpnI in the presence of ATT1. In this experiment, the cell walls of the cells were treated with bead disruption using beads of 0.5 mm size. The selection medium used is SGII-NH₄NO₃ medium containing Zeocin at 10 μg/ml. FIG. 8(B) shows C. reinhardtii (Nit1-305,cw-15) transformed with pSP124S plasmid DNA linearized with KpnI in the presence of ATT2. In this experiment, the cell walls of the cells were treated with bead disruption using beads of 0.5 mm size. The selection medium used is SGII-NH₄NO₃ medium containing Zeocin at 10 μg/ml. FIG. 8(C) shows C. reinhardtii (Nit1-305,cw-15) transformed with pSP124S plasmid DNA linearized with KpnI in the presence of ATT3. In this experiment, the cell walls of the cells were treated with bead disruption using beads of 0.5 mm size. The selection medium used is SGII-NH₄NO₃ medium containing Zeocin at 10 μg/ml. FIG. 8(D) shows C. reinhardtii (Nit1-305,cw-15) transformed with pSP124S plasmid DNA linearized with KpnI in the presence of ATT4. In this experiment, the cell walls of the cells were treated with bead disruption using beads of 0.5 mm size. The selection medium used is SGII-NH₄NO₃ medium containing Zeocin at 10 μg/ml.

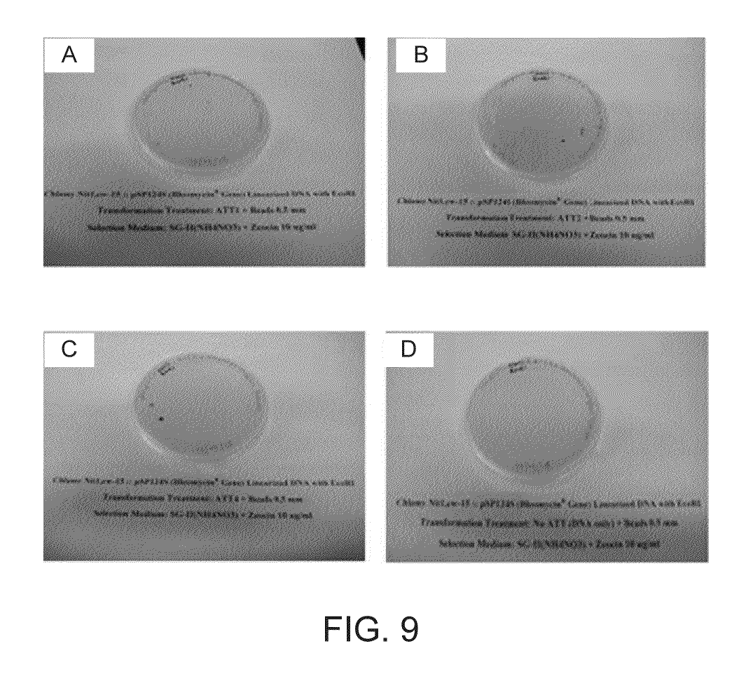

FIG. 9(A) shows C. reinhardtii (Nit1-305,cw-15) transformed with pSP124S plasmid DNA linearized with EcoRI in the presence of ATT1. In this experiment, the cell walls of the cells were treated with bead disruption using beads of 0.5 mm size. The selection medium used is SGII-NH₄NO₃ medium containing Zeocin at 10 μg/ml. FIG. 9(B) shows C. reinhardtii (Nit1-305,cw-15) transformed with pSP124S plasmid DNA linearized with EcoRI in the presence of ATT2. In this experiment, the cell walls of the cells were treated with bead disruption using beads of 0.5 mm size. The selection medium used is SGII-NH₄NO₃ medium containing Zeocin at 10 μg/ml. FIG. 9(C) shows C. reinhardtii (Nit1-305,cw-15) transformed with pSP124S plasmid DNA linearized with EcoRI in the presence of ATT4. In this experiment, the cell walls of the cells were treated with bead disruption using beads of 0.5 mm size. The selection medium used is SGII-NH₄NO₃ medium containing Zeocin at 10 μg/ml. FIG. 9(D) shows C. reinhardtii (Nit1-305,cw-15) transformed with pSP124S plasmid DNA linearized with EcoRI. No ATT was used in this experiment. In this experiment, the cell walls of the cells were treated with bead disruption using beads of 0.5 mm size. The selection medium used is SGII-NH₄NO₃ medium containing Zeocin at 10 μg/ml.

### Molecular Screening of Transformants

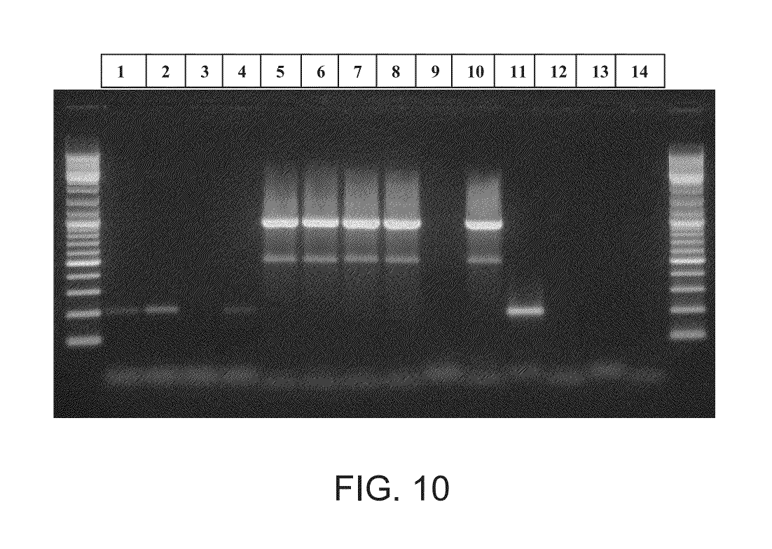

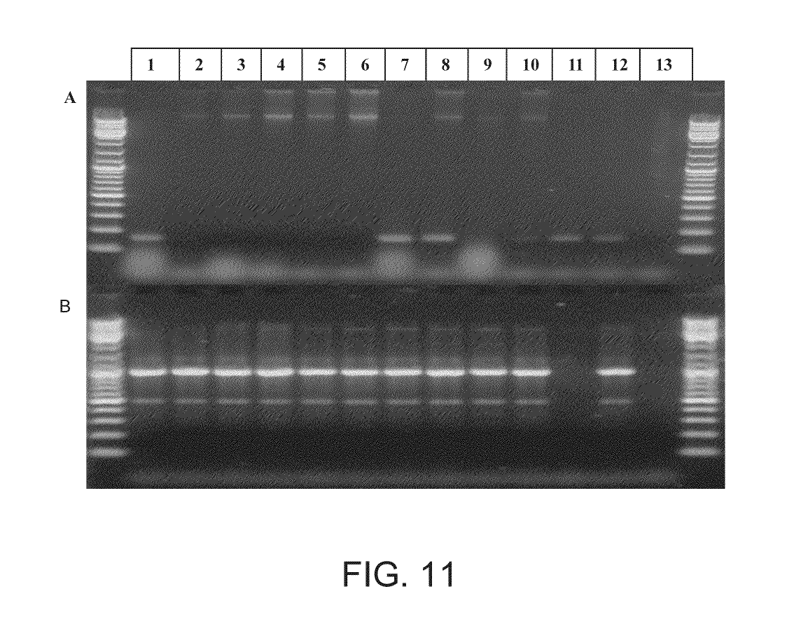

Transformed colonies were picked from the selection plates and inoculated into liquid selection media to multiply cells to be used for genomic DNA isolation. Genomic DNA from 1×10⁵ cells of untransformed algae and the transformants were isolated using DNeasy™ Blood & Tissue Kit (QIAGEN®). ReadyMix™ Taq PCR Reaction Mix with MgCl₂ (Sigma, Canada) was used to prepare 20 μl PCR reactions. Thermal cycling conditions were initial denaturation at 95° C. for 5 minutes and 40 cycles of 95° C. for 30 s, 65° C. for 30 s and 72° C. for 1.5 min. A final primer extension cycle was performed at 72° C. for 10 min. Results of PCR screening of transformants are presented in FIGS. 10 and 11. Primer sequences for each genetic constructs and to 28S rRNA gene are presented in Table 3 below.

TABLE 3
The PCR primer design information

| Gene name | Primer | |
|---|---|---|
| to amplify | name | Primer sequence |
| Algae endogenous | 28S | TCGAAATCCGCTAAGGAGTG |
| 28S rRNA | rRNA-F | (SEQ ID NO: 19) |
| (AB437257.1 and | 28S | GAGCACTGGGCAGAAATCAC |
| AF183463.1) | rRNA-R | (SEQ ID NO: 20) |
| Plasmid (pMN24): | Nit1-F3 | ACTTCCGCATCAAGATCCAC |
| nitrate reductase | | (SEQ ID NO: 21) |
| gene | Nit1-R3 | ACCTCCTCCAGCGTGTACTG |
| (XM_001696645.1) | | (SEQ ID NO: 22) |
| Plasmid (pSP124S): | Zeocin- | GCCGAGGAGCAGGACTAAC |
| bleomycin resistant | F5 | (SEQ ID NO: 23) |
| gene (construct | Zeocin- | GGAGCTAAGCTACCGCTTCA |
| sequence) | R5 | (SEQ ID NO: 24) |

FIG. 10 shows gel electrophoresis results of PCR screening of Chlamydomonas reinhardtii (nit1-305,cw15) transformed colonies. Lanes 1 and 2 show Nit gene specific PCR product from Chlamydomonas reinhardtii (nit1-305, cw15) transformants treated with ATT1 delivering the pMN24 plasmid with cellulase pretreatment. Lane 3 shows Nit gene specific PCR product from control non-transformed Chlamydomonas reinhardtii. Lane 4 shows Nit gene specific PCR product from Chlamydomonas reinhardtii (nit1-305, cw15) transformants treated with ATT1 delivering the pMN24 plasmid with lysozyme pretreatment. Lanes 5-8 are 28S rRNA internal control PCR products from cells corresponding to wells 1-4. Lane 9 shows Nit gene specific PCR product from negative control non-transformed Chlamydomonas reinhardtii. Lane 10 is 28S rRNA internal control PCR product from negative control non-transformed Chlamydomonas reinhardtii. Lane 11 shows Nit gene specific PCR product from positive control pMN24 plasmid. Lane 12 is 28S rRNA internal control PCR product from negative control pMN24 plasmid. Lane 13 is Nit gene specific PCR product from no template controls. Lane 14 is 28S rRNA internal control PCR product from no template controls.

FIG. 11 shows gel electrophoresis results of the PCR screening of transformant colonies resulted from transformation of C. reinhardtii (Nit1-305,cw-15) mutant with pSP124S (Bleomycin resistant plasmid linearized using KpnI restriction enzyme) with no pretreatment. Panel A. presents the PCR products amplified using the Zeocin specific primers (155 by expected product size). Lane 1-5 are PCR products from five colonies transformed by DNA/ATT1. Lane 1 is positive. Lane 6 is negative control Zeocin specific PCR product from control non-transformed Chlamydomonas reinhardtii. Lanes 7 and 8 show PCR products from two transformants obtained from DNA/ATT2. Lane 7 and 8 are positive. Lane 9 shows PCR product from a transformant obtained from using DNA/ATT3. Lane 10 shows PCR product from a transformant obtained using DNA/ATT4. Lane 11 is internal control PCR product from positive control pMN24 plasmid. Lane 12 is internal control PCR product from positive control pMN24 plasmid spiked into Chlamydomonas reinhardtii DNA. Lane 13 is internal control PCR product from negative no template control. Panel B. presents the products amplified by C. reinhardtii 28SrRNA gene specific primers (˜1000 by expected product sized). Wells on both the gels (A and B) are loaded with the same order of the samples. Transformation was successful even when the cells were not treated with any of the cell wall degrading enzymes.

In some experiments, the inventor observed that the transformed algal cells were able to grow for multiple generations on the selection media, suggesting that the algal cells were stably transformed. It is possible that the DNA molecules introduced into the algal cell have been integrated into the nuclear or organelle genome of the algal cell through homologous or non-homologous recombination or some other means. Additionally or alternatively, it is also possible that the DNA molecules introduced into the algal cell have remained in the algal cell as episomes.

### Example 5

Example 5 is a protocol for transforming an algal cell (e.g., Chlamydomonas reinhardtii) according to an example embodiment of the invention.
1. Grow the algal cells in 9×100 ml SGII+NH₄NO₃ medium until they reach 1×10^{6/ml or OD=}0.6-0.8 at 680 nm.
2. Spin down the cells at moderate speed (2500 rpm for 10 min in GSA rotor type) in 50 ml falcon tubes and then add the cells into one tube (Total number of cells=9×10⁸).
3. Treat the algal cells with an enzyme to disrupt the cell walls. Follow the lysozyme treatment protocol or cellulase treatment protocol as described in Example 4. In order to stop the enzymatic reaction, wash the cells twice with distilled water. This will remove enzyme(s) used for cell wall degradation.
4. Resuspend the cells in 9 ml SGII+NH₄NO₃ medium (in case of transformation using pMN24 plasmid containing the nitrate reductase gene, resuspend the cells in SGII+KNO₃ medium). The total cell numbers would be 1×10⁸/ml. Use 300 μl of cell suspension (3×10⁷ number of cell) in each treatment.
5. Transfer 300 μl cell suspension to 15 ml Falcon tube containing the required size of beads (300 mg) and/or whiskers (40 μl from 50 mg/ml stock) as according to the treatment.
6. Just before moving to the next step, prepare DNA and ATT reaction mixture as follows:

| | Total | 100 μM | | Total |
|---|---|---|---|---|
| | DNA | ATT peptide | H₂O | volume |
| Name | 1.0 μg (μl) | volume (μl) | (μl) | (μl) |
| ATT1 = 13.53 ng | 10 | 4.76 | 5.24 | 20 |
| ATT2 = 40.13 ng | 10 | 2.87 | 7.13 | 20 |
| ATT3 = 80.22 ng | 10 | 3.57 | 6.43 | 20 |
| ATT4 = 47.85 ng | 10 | 3.36 | 6.64 | 20 |
| DNA Control | 10 | 0 | 10 | 20 |
| Negative | 0 | 0 | 20 | 20 |
| Control | | | | |

7. Add the reactions to the mixture of the cells, beads (as per treatment).
8. Vortex the suspensions for 15 s.
9. Add 10 ml SGII+NH₄NO₃ (in case of transforming using pMN24 plasmid containing nitrate reductase gene, resuspend the cells in SGII+KNO₃) medium and incubate for overnight by shaking at 100 rpm to allow for cell recovery. In case of treatments using beads, transfer the cell suspension into a fresh Falcon tube leaving the beads separated before the next step.
10. Pellet the cells by centrifugation for 5 minutes at 3000 rpm.
11. Resuspend the cells (transformed and wild type without any treatment as negative control) in 1.5 ml of melted selection agar (0.5%) medium (maintained at 45° C. in water bath) to the tube. Mix by gentle pipetting, and pour the entire volume onto the plate containing 1.5% agar selection medium.
Also resuspend wild type cells for viability check, in 1.5 ml of melted non-selection agar (0.5%) medium (maintained at 45° C. in water bath) to the tube. Mix by gentle pipetting, and pour the entire volume onto the plate containing 1.5% non-selection medium.
12. Allow the agar to cool and harden, wrap the plates with parafilm to prevent desiccation, and incubate the plates in a 24+1° C. growth incubator under a 16/8 hr day/night cycle.
13. Transformant colonies are expected to be visible after 6-8 days of growth.

### Example 6

Gel Mobility Shift Assay of Peptide Plasmid DNA Complex Formation

Purified linearized plasmid DNA (e.g., 200 ng of a 4566-bp plasmid) was mixed with different concentrations of each peptide according to the calculated peptide DNA weight ratios of 1:1, 2:1, 3:1, 4:1 up to 10:1 or a 1/10 dilution of the previously stated ratios until a complete shift was observed in the plasmid DNA during electrophoresis. The DNA was prepared to a final concentration of a 200 ng/μl in sterile water. Each reaction had a final volume of 20 μl and was incubated for 25 min for complex formation and subjected to electrophoresis on 1% agarose gel stained with ethidium bromide.

Results The ATT1-4 peptides were tested for their ability to non-covalently bind to nucleic acids. Non-covalent attachment was tested using a gel mobility shift assay. The gel mobility shift assay was used to determine the minimum peptide concentration needed bind to plasmid DNA and cause it to shift during electrophoresis. Based on a peptide-DNA charge ratio calculation increasing concentrations of peptide were added to 200 ng of linearized plasmid DNA until the charge of the DNA was neutralized by the peptide causing a mobility shift in the DNA during electrophoresis in a 1% agarose gel.

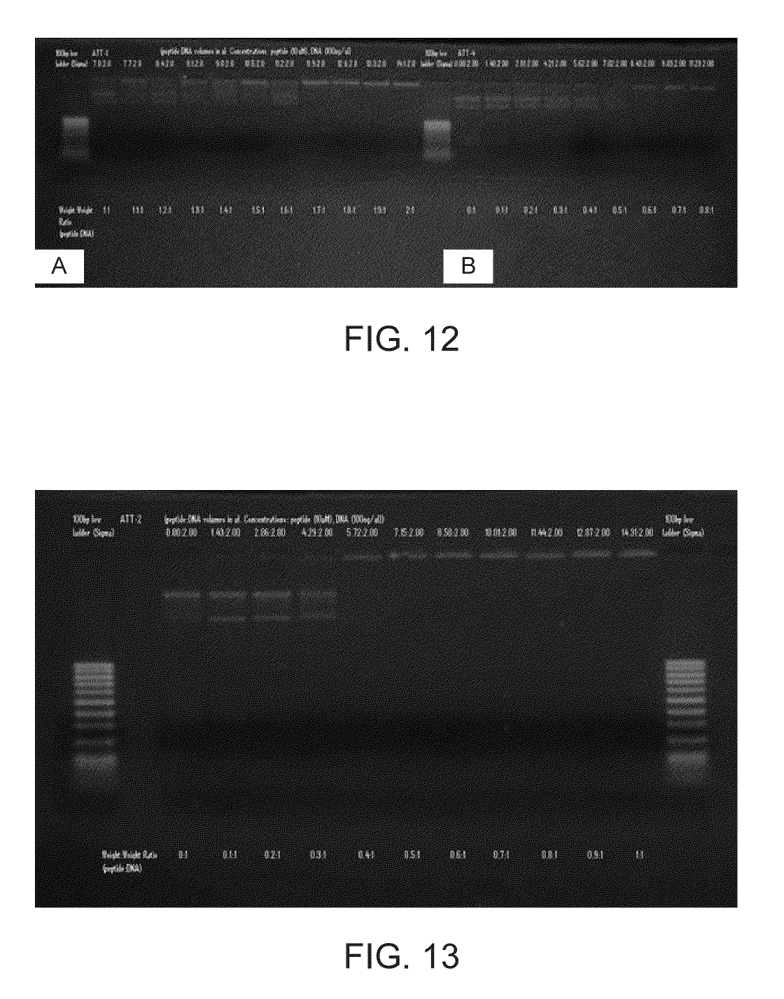

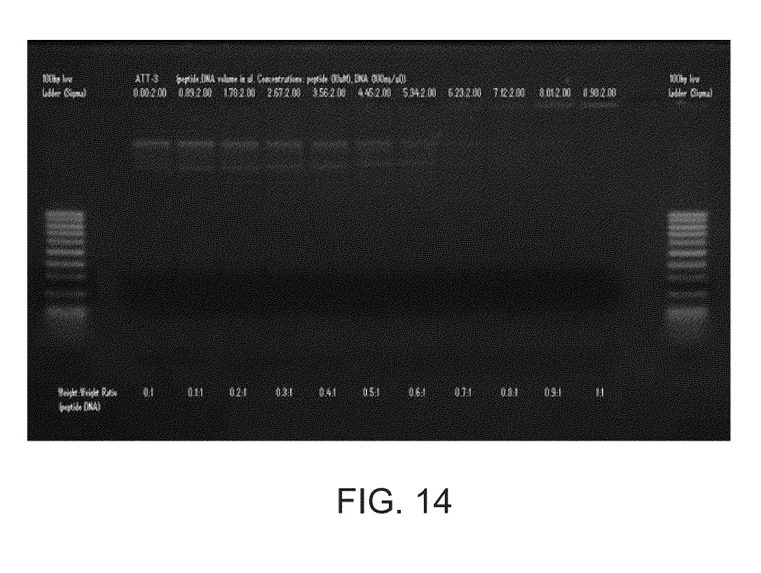

FIG. 12 is an image of 1% agarose gel showing the gel mobility shift assay results for ATT1 and ATT4. A) The shift occurred at the peptide/DNA ratio of 1.7:1 or 339 ng of ATT1. B) The shift occurred at the peptide/DNA ration of 0.6:1 or 120 ng of ATT4. FIG. 13 is an image of 1% agarose gel showing the gel mobility shift assay results for ATT2. The shift occurred at the peptide/DNA ratio of 0.4:1 or 80 ng of ATT2.FIG. 14 is an image of 1% agarose gel showing the gel mobility shift assay results for ATT3. The shift occurred at the peptide/DNA ratio of 0.9:1 or 180 ng of ATT3.

All of the peptides ATT1 to ATT4 were able to cause a shift in the DNA during electrophoresis. The shift occurred at the peptide/DNA ratio of 1.7:1 or 339 ng of ATT1. The shift occurred at the peptide/DNA ratio of 0.4:1 or 80 ng of ATT2. The shift occurred at the peptide/DNA ratio of 0.9:1 or 180 ng of ATT3. The shift occurred at the peptide/DNA ratio of 0.6:1 or 120 ng of ATT4.

In some transformation protocols, the ATT plasmid DNA complex was created by combining 4× the peptide concentration needed to cause a shift in the DNA during the gel mobility shift assay. The determined peptide concentration was scaled up from the amount need to cause a gel shift of 200 ng plasmid DNA and combined with 1.5 μg (or some other suitable amount) of plasmid DNA in a final volume of 20 μl. This mixture was incubated for 10 minutes at room temperature.

As will be apparent to those skilled in the art in the light of the foregoing disclosure, many alterations and modifications are possible in the practice of this invention without departing from the spirit or scope thereof

### REFERENCES

1. Greenwell, H. C., et al., Placing microalgae on the biofuels priority list: a review of the technological challenges. Journal of The Royal Society Interface, 2010. 7(46): p. 703-726.
2. Mehta, S. K. and J. P. Gaur, Use of Algae for Removing Heavy Metal Ions From Wastewater: Progress and Prospects. Critical Reviews in Biotechnology, 2005. 25(3): p. 113-152.
3. Becker, W., Microalgae in Human and Animal Nutrition. Handbook of Microalgal Culture. 2007: Blackwell Publishing Ltd. 312-351.
4. Specht, E., S. Miyake-Stoner, and S. Mayfield, Micro-algae come of age as a platform for recombinant protein production. Biotechnology Letters, 2010. 32(10): p. 1373-1383.
5. Giordano, M., J. Beardall, and J. A. Raven, CO₂ CONCENTRATING MECHANISMS IN ALGAE: Mechanisms, Environmental Modulation, and Evolution. Annual Review of Plant Biology, 2005.56(1): p. 99-131.
6. Dismukes, G. C., et al., Aquatic phototrophs: efficient alternatives to land-based crops for biofuels. Current Opinion in Biotechnology, 2008. 19(3): p. 235-240.
7. Gouveia, L. and A. Oliveira, Microalgae as a raw material for biofuels production. Journal of Industrial Microbiology &amp; Biotechnology, 2009. 36(2): p. 269-274.
8. Service, R. F., ExxonMobil Fuels Venter's Efforts To Run Vehicles on Algae-Based Oil. Science, 2009. 325(5939):p. 379.
9. Cardi, T., P. Lenzi, and P. Maliga, Chloroplasts as expression platforms for plant-produced vaccines. Expert Review of Vaccines, 2010. 9(8): p. 893-911.
10. Rasala, B. A., et al., Production of therapeutic proteins in algae, analysis of expression of seven human proteins in the chloroplast of Chlamydomonas reinhardtii. Plant Biotechnology Journal, 2010. 8(6): p. 719-733.
11. Day, A., et al., Studies on the maintenance and expression of cloned DNA fragments in the nuclear genome of the green alga Chlamydomonas Reinhardtii. Physiologia Plantarum, 1990. 78(2): p. 254-260.
12. Shimogawara, K., et al., High-Efficiency Transformation of Chlamydomonas reinhardtii by Electroporation. Genetics, 1998. 148(4): p. 1821-1828.
13. Kumar, S. V., et al., Genetic transformation of the green alga—Chlamydomonas reinhardtii by Agrobacterium tumefaciens. Plant Science, 2004. 166(3): p. 731-738.
14. Pooga, M. and U. Langei, Synthesis of cell-penetrating peptides for cargo delivery. Methods Mol Bioi, 2005. 298: p. 77-89.
15. Futaki, S., et al., Arginine-rich Peptides. AN ABUNDANT SOURCE OF MEMBRANE-PERMEABLE PEPTIDES HAVING POTENTIAL AS CARRIERS FOR INTRACELLULAR PROTEIN DELIVERY. J. Bioi. Chem., 2001. 276(8): p. 5836-5840.
16. Brooks, H., B. Lebleu, and E. Vives, Tat peptide-mediated cellular delivery: Back to basics. Advanced Drug Delivery Reviews, 2005. 57(4 SPEC. ISS.): p. 559-577.
17. Wender, P. A., et al., The design, synthesis, and evaluation of molecules that enable or enhance cellular uptake: Peptoid molecular transporters. Proceedings of the National Academy of Sciences, 2000. 97(24): p. 13003-13008.
18. Herce, H. D. and A. E. Garcia, Cell Penetrating Peptides: How Do They Do It? Journal of Biological Physics, 2008: p. 1-12.
19. Vives, E., P. Brodin, and B. Lebleu, A Truncated HIV-1 Tat Protein Basic Domain Rapidly Translocates through the Plasma Membrane and Accumulates in the Cell Nucleus. J. Bioi. Chem., 1997. 272(25): p. 16010-16017.
20. Derossi, D., et al., The third helix of the Antennapedia homeodomain translocates through biological membranes. J. Bioi. Chem., 1994. 269(14): p. 10444-10450.
21. Desha yes, S., et al., Structural polymorphism of two CPP: An important parameter of activity. Biochimica et Biophysica Acta-Biomembranes, 2008. 1778(5): p. 1197-1205.
22. Hawiger, J., Cellular import of functional peptides to block intracellular signaling. Current Opinion in Immunology, 1997. 9(2): p. 189-194.
23. Lin, Y.-Z., et al., Inhibition of Nuclear Translocation of Transcription Factor NF-B by a Synthetic Peptide Containing a Cell Membrane-permeable Motif and Nuclear Localization Sequence. J. Bioi. Chem., 1995. 270(24): p. 14255-14258.
24. Liu, K. Y., et al., Identification of a functionally important sequence in the cytoplasmic tail of integrin beta 3 by using cell-permeable peptide analogs. Proceedings of the National Academy of Sciences of the United States of America, 1996. 93(21): p. 11819-11824.
25. Morris, M. C., et al., A new peptide vector for efficient delivery of oligonucleotides into mammalian cells. Nucl. Acids Res., 1997. 25(14): p. 2730-2736.
26. Morris, M. C., et al., A peptide carrier for the delivery of biologically active proteins into mammalian cells. Nat Biotech, 2001. 19(12): p. 1173-1176.
27. Pujals, S., et al., all-D proline-rich cell-penetrating peptides: a preliminary in vivo internalization study. Biochemical Society Transactions, 2007. 035(4): p. 794-796.
28. Fernandez-Carneado, J., et al., Potential peptide carriers: Amphipathic proline-rich peptides derived from then-terminal domain of gamma-zein. Angewandte Chemie-international Edition, 2004. 43(14): p. 1811- 1814.
29. Mana, M., et al., Cellular uptake of S413-PV peptide occurs upon conformational changes induced by peptide-membrane interactions. Biochimica et Biophysica Acta (BBA)—Biomembranes, 2006. 1758(3): p. 336-346.
30. Pooga, M., et al., Cell penetration by transportan. FASEB Journal, 1998. 12(1): p. 67-77.
31. Elmquist, A., et al., VE-cadherin-derived cell-penetrating peptide, pVEC, with carrier functions. Exp Cell Res, 2001. 269(2): p. 237-44.
32. Krauss, U., et al., In vitro gene delivery by a novel human calcitonin {hCT}-derived carrier peptide. Bioorganic-and-Medicinal-Chemistry-Letters, 2004. 14(1): p. 51-54.
33. Lundberg, P., et al., Cell membrane translocation of the N-terminal {1-28} part of the prion protein. Biochemical and Biophysical Research Communications, 2002. 299(1): p. 85-90.
34. Andersen, R. A. 2005. Algal culturing techniques. Elsevier Academic Press, London. Pp. 435-436.
35. Gorman, D. S., and R. P. Levine (1965). Cytochrome f and plastocyanin: their sequence in the photosynthetic electron transport chain of Chlamydomonas reinhardii. Proc. Natl. Acad. Sci. USA 54, 1665-1669.
36. Hunt, R. W., Chinnasamy, S., Bhatnagar, A. and Das, K. C. 2010. Effect of biochemical stimulants on biomass productivity and metabolite content of the microalgae
37. Kindle, K. L. 1990. High-frequency nuclear transformation of Chlamydomonas reinhardtii. Proc. Natl. Acad. Sci. USA, 87:1228-1232.
38. Lee, Y.-K. and Zhang, D.-H. 1999. Production of astaxanthin by Haematococcus. In: Z. Cohen (Ed.), Chemicals from Microalgae, Taylor and Francis, London: 173-190.
39. Lumbreras, V., Stevens, D. R. and Purton, S. 1998. Efficient foreign gene expression in Chlamydomonas reinhardtii mediated by an endogenous intron. The Plant Journal. 14(4):441-447.
40. McCaffrey et al. 2010. Use of plant growth regulators to enhance algae growth for the production of added value products. Patent Application Publication No.: US 2010/0210002A1
41. Sanger, R. And Granick, S. 1953. Nutritional studies with Chlamydomonas reinhardtii. Ann. N.Y. Acad. Sci. 56:831-838.
   All references mentioned herein, including those references listed above, are hereby each specifically incorporated by reference herein.

What is claimed is:

1. A method for introducing a cargo molecule into an algal cell, the method comprising:
preparing a composition comprising the cargo molecule and a cell penetrating peptide (CPP); and
exposing the algal cell to the composition.

2. A method according to claim 1 comprising treating the algal cell to disrupt a cell wall of the algal cell.

3. A method according to claim 2 wherein the treating step comprises exposing the algal cells to an enzyme which degrades the cell wall of the algal cell.

4. A method according to claim 3 wherein the enzyme is autolysin, lysozyme or cellulase.

5. A method according to claim 2 wherein the treating step comprises adding beads to the composition containing the algal cells and agitating the composition.

6. A method according to claim 1 wherein the cargo molecule is a nucleic acid molecule, a polypeptide, a lipid, a carbohydrate, a drug, or an imaging agent.

7. A method according to claim 1 wherein the cargo molecule comprises a detectable marker, a selectable marker, or a reporter gene.

8. A method according to claim 1 wherein the cargo molecule comprises a gene of interest which encodes a protein of interest.

9. A method according to claim 8 wherein the cargo molecule comprises a promoter which is operably linked to the gene of interest for driving the expression of the gene of interest in the algal cell.

10. A method according to claim 8 wherein the cargo molecule comprises both the gene of interest and a selectable marker.

11. A method according to claim 1 wherein the step of preparing the composition comprises covalently coupling the cargo molecule to the cell penetrating peptide.

12. A method according to claim 1 wherein the step of preparing the composition comprises associating the cargo molecule to the cell penetrating peptide in a non-covalent fashion.

13. A method according to claim 1 wherein the CPP is Transportan (SEQ ID NO: 11), Penetratin (SEQ ID NO: 4), an HIV Tat fragment, or a polyarginine, or a variant thereof.

14. A method according to claim 13 wherein the HIV Tat fragment is Tat (48-57) (SEQ ID NO: 3) or Tat (49-57) (SEQ ID NO: 1).

15. A method according to claim 13 wherein the polyarginine is (Arg)7 (SEQ ID NO: 14), (Arg)8 (SEQ ID NO: 15), (Arg)9 (SEQ ID NO: 16), (Arg)10 (SEQ ID NO: 17), or (Arg)11 (SEQ ID NO: 18).

16. A method according to claim 1 wherein the algal cell is an algal cell of the species Chlamydomonas reinhardtii, Spirulina palatensis, Botryococcus braunii, Chiarella sp., or Chiarella protothecoides.

17. A method for transforming an algal cell, the method comprising:
preparing a composition comprising a nucleic acid molecule and a cell penetrating peptide; and
exposing the algal cell to the composition.

18. A composition for introducing a cargo molecule into an algal cell, the composition comprising a cargo molecule and a cell penetrating peptide (CPP).

19. A composition according to claim 18 wherein the CPP is Transportan (SEQ ID NO: 11), Penetratin (SEQ ID NO: 4), an HIV Tat fragment, or a polyarginine.

20. A transformed algal cell produced according to the method of claim 17.


SEQUENCE LISTING (24 sequences)

SEQ ID NO: 1 - protein, 9 aa, artificial sequence
  synthetic construct
RKKRRQRRR

SEQ ID NO: 2 - protein, 18 aa, artificial sequence
  synthetic construct
RKKRRQRRRRKKRRQRRR

SEQ ID NO: 3 - protein, 10 aa, artificial sequence
  synthetic construct
GRKKRRQRRR

SEQ ID NO: 4 - protein, 16 aa, artificial sequence
  synthetic construct
RQIKIWFQNRRMKWKK

SEQ ID NO: 5 - protein, 16 aa, artificial sequence
  synthetic construct
AAVALLPAVLLALLAP

SEQ ID NO: 6 - protein, 15 aa, artificial sequence
  synthetic construct
VTVLALGALAGVGVG

SEQ ID NO: 7 - protein, 27 aa, artificial sequence
  synthetic construct
GALFLGFLGAAGSTMGAWSQPKKKRKV

SEQ ID NO: 8 - protein, 21 aa, artificial sequence
  synthetic construct
KETWWETWWTEWSQPKKKRKV

SEQ ID NO: 9 - protein, 18 aa, artificial sequence
  synthetic construct
VRLPPPVRLPPPVRLPPP

SEQ ID NO: 10 - protein, 21 aa, artificial sequence
  synthetic construct
ALWKTLLKKVLKAPKKKRKVC

SEQ ID NO: 11 - protein, 27 aa, artificial sequence
  synthetic construct
GWTLNSAGYLLGKINLKALAALAKKIL

SEQ ID NO: 12 - protein, 18 aa, artificial sequence
  synthetic construct
LLIILRRRIRKQAHAHSK

SEQ ID NO: 13 - protein, 28 aa, artificial sequence
  synthetic construct
MANLGYWLLALFVTMWTDVGLCKKRPKP

SEQ ID NO: 14 - protein, 7 aa, artificial sequence
  synthetic construct
RRRRRRR

SEQ ID NO: 15 - protein, 8 aa, artificial sequence
  synthetic construct
RRRRRRRR

SEQ ID NO: 16 - protein, 9 aa, artificial sequence
  synthetic construct
RRRRRRRRR

SEQ ID NO: 17 - protein, 10 aa, artificial sequence
  synthetic construct
RRRRRRRRRR

SEQ ID NO: 18 - protein, 11 aa, artificial sequence
  synthetic construct
RRRRRRRRRRR

SEQ ID NO: 19 - DNA, 20 bp, artificial sequence
  synthetic construct
tcgaaatccgctaaggagtg

SEQ ID NO: 20 - DNA, 20 bp, artificial sequence
  synthetic construct
gagcactgggcagaaatcac

SEQ ID NO: 21 - DNA, 20 bp, artificial sequence
  synthetic construct
acttccgcatcaagatccac

SEQ ID NO: 22 - DNA, 20 bp, artificial sequence
  synthetic construct
acctcctccagcgtgtactg

SEQ ID NO: 23 - DNA, 19 bp, artificial sequence
  synthetic construct
gccgaggagcaggactaac

SEQ ID NO: 24 - DNA, 20 bp, artificial sequence
  synthetic construct
ggagctaagctaccgcttca